(19) United States
(12) Patent Application Publication
(10) Pub. No.: US 2010/0082048 A1
(43) Pub. Date: Apr. 1, 2010

(54) PROSTHESIS FOR ANASTOMOSIS

(76) Inventor: Luiz Gonzaga Granja Filho, Recife (BR)

Correspondence Address: VOLPE AND KOENIG, P.C., UNITED PLAZA, SUITE 1600, 30 SOUTH 17TH STREET, PHILADELPHIA, PA, 19103, US

(21) Appl. No.: 12/303,540

(22) PCT Filed: Jun. 6, 2007
(86) PCT No.: PCT/BR2007/000141

§ 371 Date: Mar. 30, 2009

(30) Foreign Application Priority Data
Jun. 6, 2006 (BR) PI 0602380-0

(51) Int. Cl. A61B 17/08

(52) U.S. Cl. 606/153

(57) ABSTRACT

Prosthetic devices are provided that are used in end-to-side, end-to-end and side-to-side anastomosis without clamping and sutureless, without clamping and with suture, with clamping and sutureless, and/or with clamping and with suture, where the graft is inserted under the light of prosthesis or in at least one of the intraluminal portions of the prosthesis tubular member. The prosthesis can be produced in varied shapes and sizes to accommodate varied sizes and types of grafts, and also can be formed by two halves that can be joined by pressure, bolts or by a rocker portion, and can be made of any proper material for surgical use, such as titanium, stainless steel, nitinol, pyrolitic carbon, silicon, biodegradable materials, or any other biocompatible and inert materials.

### FIELD OF INVENTION

The present invention relates in a general manner to anastomotic devices and more specifically to a prosthetic device that allows anastomosis without clamping and sutureless, or with quick clamping and sutureless, where a vascular graft, or any other, is inserted in the prosthesis lumen and reversed by jacketing to cover part of the prosthesis, that will remain inside the graft (vein, artery or tissue) and is fixed on the tubular member of prosthesis by a round point, or other safe method. The prosthesis flange has a plurality of spaced openings in its peripheral part, allowing the prosthesis to be sutured out of anastomosis, just for its fixation, on tissue vein, artery or any other organ, so eliminating one of the main causes of obstruction of anastomosis, which is the introduction of foreign bodies inside the lumen and especially by eliminating clamping that is the main factor and responsible for the occurrence of thromboembolic accidents and lacerations of friable tissues.

### DESCRIPTION OF THE PRIOR ART

A prior art presents several trials provide solutions for anastomotic devices projected to correct vascular abnormalities, which present the following typical features:

The North-American U.S. Pat. No. 3,254,650, of Jun. 7, 1966, describes a method and devices to execute anastomosis procedures by applying with adhesive two separated connectors in a body member and removing this body member portion contained among the connectors, joining the said connection devices for joining the remaining portions of the body member.

The U.S. Pat. No. 3,265,069, of Aug. 9, 1966, describes devices or instruments for use by surgeons in reunion of body ducts, which in the course of operations were separated. The instruments comprise a pair of elongated similar elements and articulatedly connected, in an intermediary manner, and with an support for finger retention in a distal end, comprising a generally cylindrical shape with a cylindrical channel that passes through it in the other distal end, in order to receive tubular body ducts kept by the instrument while the body ducts are reconnected.

U.S. Pat. No. 3,774,615, of Nov. 27, 1973, describes a device to connect the end of interrupted tubular organs without sewing, comprising a connecting ring on which the end of the interrupted organ are pulled, the ring is preferably locked up by a fixation resource. The ring and fixation resource are made of inert material, and preferably a hydrophile gel that can be dilated until its equilibrium or can be a hydrogel incompletely dilated, which is submitted to additional dilatation where it is applied. The connecting ring can be supplied with a groove and can be placed in a ring shaped fixation resource and kept there joining it to the fixation resource in the groove or simply kept by a screw. Two connection rings can also be used and kept joined by a coupling member.

The document U.S. Pat. No. 4,366,819, of Jan. 4, 1983, describes an anastomotic joint for surgery with a graft of coronary artery deviance comprising a mounting of four elements including a cylindrical tube with at least one locking indentation of ring flange in one influx end and a plurality of grooves of locking ring in a flow end; a ring flange with a central opening and a plurality of long and short spigots, the long spigots are engaged in the locking indentation, with a graft engaged among them; a fixation ring with a central opening and a plurality of spigots positioned around the opening; and a locking ring with a opening with a plurality of locking ring edges for engaging with the locking ring grooves. In surgical implants, an aortic wall with a hole engages between the ring flange and the fixation ring and is kept in this position by spigots of the fixation ring, and the four elements engage together forming an integral anastomotic joint. A first alternate modality includes an anastomotic joint of three elements with a combination of fixation ring and locking ring. A second alternate modality includes an anastomotic joint of four elements with a slightly jolted end in a influx end, exposing the graft material in the anastomotic “ostium”.

Other prior arts are equally mentioned, base don some information of “The Cardiothoracic Surgery Network”. The “Symmetry Aortic Connector System”, developed by St. Jude Medical, is a connector made with nitinol, selected by vein diameter with an adventitia removed to allow adjust of the connector and to prevent its displacement by the blood current. Then, the device may make an angle of 90° with the aorta. Among the disadvantages, there is the fact that it can be used only in extreme cases due to the difficult usage of this technique; it did not obtain a satisfactory result in many surgeries and it is being drowned out of market by the manufacturer; it is not applicable in calcified aorta; presents suture; presents contact with blood flow (foreign body); it does not widen the anastomosis area (restrictive anastomosis); performs only one anastomosis at a time; it is a product restrict to end-to-side anastomosis; a great mobilization of the venous graft occurs, damaging it, and can eventually form thrombus; there is a risk of perforation of the posterior wall of aorta; and the adventitia is removed (most resistant vascular layer).

Other known device is the PAS-Port™ System, a device used in 3 steps, and the vein wall is mounted over the device and is manually reversed on it, by tool and adapted to aorta with a angle of 90°. The method alerts that the surgeon shall select with due care the point of aorta and the vein size. The device is made of stainless steel and is available in only one size that allows the use of veins with external diameter of 4 to 6 mm, aorta with an internal diameter of 18 mm. It is available in only one size, limiting its applicability. As disadvantages of this prior art, the device has contact with blood flow (foreign body); it does not widen the anastomosis area (restrictive anastomosis); it uses veins with external diameter of 4 to 6 mm and aorta with an internal diameter of 18 mm; it does not perform multiple nor visceral anastomosis; it performs just only end-to-side anastomosis; a great mobilization of the used biological graft occurs, damaging its inner layer, which generates the formation of thrombus; there is a big risk of kinking at the origin (angle of)90° and risk of posterior wall perforation in the aorta at the moment the device is introduced under its light; the suture is substituted with disadvantages by stainless steel (9 pins, distant among them, maximizing the risk of bleeding).

Also as prior art, there is the CorLink Device, currently commercialized by Ethicon/Johnson & Johnson, that allows the creation of anastomosis between the ascending aorta and a saphenous vein segment. Aortic Anastomotic Device (AAD) is a self-expanded device with extra luminal nitinol constituted by a de um central cylinder with five interconnected elliptical arches and 2 groups of 5 pins in the end portion of the cylinder. The pins, after the eversion of venous walls in the device, fix the aggregate penetrating into the venous graft wall. A blade makes an opening in the wall of aorta and permits the coupling of AAD, which also fix the wall of aorta by pins. With this device: it poses a serious risk of bleeding, especially in friable aortas, thin, calcified or fibrous, restricting its applicability, also with risks, even in aortas with normal walls; in small gauge anastomosis, there is a risk of thrombosis, hyperplasia, intimal proliferation and fibrosis (reaction to foreign body type in origin of anastomosis) with consequent stenosis resulting in occlusion of anastomosis; sutures are used in some cases; there is cases of infarction caused by equipment; there is a recurring need of re-operations in patients; the device presents contact with blood flow (foreign body); it is not flexible; it does not multiple anastomosis; an inadequate mobilization of venous graft occurs, and can cause damage to its intimal layer, it could form thrombus; it is used only in extreme cases because it is a technique of complex usage; the suture is substituted by stainless steel in contact with blood flow.

Another known device is the St Jude Distal Connector that consists of a stainless steel clip mounted on a catheter, comprising a balloon for subsequent expansion and connector mounting. The catheter is introduced backward from the end, by doing a small hole in the anastomosis site, the clip fixes the vein in the hole, the catheter goes to coronary and releases the connector. The catheter is removed and a suture is done in side-to-side anastomosis. With St Jude Distal Connector, occurrence of leakage problems were detected in 20% of the used connectors; the use of a metallic clip requires due care for handling to avoid distortion in the anastomosis; late angiographies reveal smaller circular diameter of anastomosis made with o St. Jude Distal Connector, when compared to controls made with conventional suture; there is remarkably risk of bleeding and the graft is very mobilized, and lacerations can occur in its inner layer, allowing the formation of thrombus.

The HeartFlo™ is a multi-suture instrument for anastomosis with wires automatically applied in end-to-side and side-to-side anastomosis. The surgeon manually ties the suture wires (10 wires) and concludes the anastomosis similarly to the traditional process. Besides of being a product of complex handling, it makes suture in anastomosis (keeping the undesirable foreign body in the internal origin of the anastomosis) and is restricted to end-to-side and side-to-side anastomosis. There is also an excessive mobilization of graft, and can cause lesions in its intimal layer, which would be the inductor that forms the thrombus.

Another technique and known device is the Solem Graft connector, produced by the Swedish company Jomed. It is constituted by a stent made of nickel and titanium coated with polytetrafluorethylene used to connect the internal thoracic artery the left anterior descending coronary artery. The results has not been satisfactory, because it poses risk of bleeding; there is also an excessive mobilization of graft, probably damaging intimal layers, allowing the formation of thrombus; it is not flexible, by this fact, causes trauma to grafts; it does not make multiple anastomosis, at a single time; presents contact with blood flow (foreign body); and is frequent the need of-operations.

The Magnetic Vascular Positioner System is produced by Ventrica and comprises 4 magnetic rings and the anastomosis is processed by magnetic attraction of 4 ports. However, initial experimental results demonstrate leakage, also a undesired contact of materials with blood flow. On the other hand, it is necessary to be careful to avoid the capitation of excess of tissue among the magnets. With this system, there is also a need of suture in some cases; there is occurrence of infarction caused by equipment; and is frequent the need of-operations in patients; and also requires clamping.

Also, as a device known by the medical area, the Combined Anastomotic Device and Tissue Adhesive, developed by Grundeman & Borst group, combines micro mechanical technique with use of adhesive (glue). The use of this method can result in leakages and need traditional sutures; it is frequent the need of re-operation due to leakage/bleeding; and performs only one anastomosis at a time.

Finally, it is also experimentally practiced anastomosis assisted by laser, where the results are not different from conventional isolated sutures, because there is a need of suture in some cases; there is a risk of bleeding e leakage; and does not perform multiple anastomosis.

Even so divulged nowadays, anastomosis with damper, by insecurity, and almost totality of surgeons perform conventional sutures throughout the route of anastomosis, with an intention of avoiding leakages and bleedings, it means the use of clampers just makes the procedure more expensive, once the conventional suture is also applied.

In short, the conventional anastomosis, with clamping and with suture, standardized in 1906 by Alexis Carrel, remains the first choice for any type of anastomosis and organs to be anastomosed.

With an expectation of changing the current situation, the Brazilian patent no. PI 9706197-2, describes and claims a prosthesis for vascular anastomosis, or in any other organ or tissue, without the use of clamping and sutureless, solving, in an elegant and efficient manner, the limitations inherent to prosthesis of the above mentioned prior art, when used in vascular anastomosis performed, mainly in thin aortas, calcified and friable; or in any other application where a clamping of a vein or artery can pose excessive trauma for conditions of a given patient. The prosthesis that is subject of that request allows the embodiment of fast and safe anastomosis, without obstruction of vein or artery lumen of which anastomosis is made, also allows anastomosis in tissues, veins or arteries in bad conditions and never would accept a clamping used in conventional anastomosis. This is achieved by a generally cylindrical shaped prosthesis with a flange orthogonally extending from its external side wall, in a point in the prosthesis length between its ends; the referred flange has openings distributed around its surface. The description of the usage method and specific construction of the prosthesis is presented in the drawings of the descriptive report of that request, as well as the document C19706197-2, Certificate of Addition of the first.

Although these anastomotic devices can be presented as suited to the purposes for which they were projected, they are not so suited for the purposes of the present invention, as described herein below.

### SUMMARY OF THE INVENTION

A present invention relates to variations of the currently known anastomotic devices, in order to allow an anastomosis without clamping and sutureless, or with quick clamping and sutureless in organs with normal walls or where clamping does not represent a major risk (intestines etc), where at least one vascular graft, or any other, is inserted in the prosthesis lumen and reversed by jacketing to cover part of the prosthesis, and is fixed in the tubular member of prosthesis by a round point, or any other safe method. The tubular member of prosthesis is contiguous to the flange, which can or not have openings, allowing the sewing of flange just for its fixation, outside of the tissue, vein, artery or tubular organ to eliminate the contact with foreign bodies inside the anastomosis. The prosthesis can also have varied sizes and shapes to simultaneously accommodate grafts of varied sizes and types.

One objective of the present invention is to provide an anastomotic device with more than one tubular member, allowing the insertion of one more graft, of different types and gauges, separately or previously joined among them, in the same prosthesis (for example, anastomotic trunk, resulted from the junction of multiple grafts ends, in the desired extension, being them autologs, homologs, heterologs and/or synthetic, Dacron, PTFE, Gor-Tex type).

Another objective of the present invention is to provide a prosthesis with flanges of different shapes and intraluminal portions of different sizes to permit anastomosis side-to-side among grafts of different gauges and small gauges. Also, another objective of the present invention is to provide an anastomotic device with a plurality of chamfers placed at the peripheral part of the flange, in order to avoid that the interposition of the suture wire handle to the light of prosthesis.

Other objective of the present invention is to provide an anastomotic device with two or more halves that join by pressure, by fixation devices, by rocker movements, and so on, in order to facilitate grafts eversion to cover the intraluminal portion of the device and to allow the use of grafts of variable diameters with a single device which diameter increases or diminishes.

Other features and additional objectives of the present invention will become apparent from the following descriptions. These features will be described at sufficiently detailed levels to allow the technicians of the subject matter to implement the invention. Also, it is understood that other features can be used and structural changes can be made without leaving the scope of the invention. In the accompanying drawings, like reference numbers indicate identical or like parts throughout the several views.

Therefore, the following detailed description should not be taken as limiting the scope of the present invention which is defined by the appended claims.

### BRIEF DESCRIPTION OF THE DRAWINGS

The present invention may be understood more completely by reference to the following description and appended drawings, supported by examples, in which:

FIG. 1 is a perspective view of a prosthesis with more than one intraluminal portion that can comprise the equal or different extension and diameters, in order to allow the junction of two or more grafts, of equal or different types and gauges.

FIG. 2 illustrates a modality of anastomotic trunk (junction of two or more grafts, equal or different)

FIG. 3A is an upper plan view of the flange with chamfers, and anchored suture wires, in order to impede the interposition of the wire handle to the light of prosthesis.

FIG. 3B is an upper plan view of a flange embodiment without holes.

FIG. 3C is an upper plan view of a flange embodiment without holes of FIG. 3B with suture wires passed through.

FIG. 3D is a perspective view of flange embodiment without holes of FIG. 3B, in order to illustrate the flange edge to anchor suture wires handle.

FIG. 4A is an upper view of prosthesis with elliptical and short intraluminal portions for side-to-side anastomosis in any tubular organs of any gauge.

FIG. 4B is a perspective view of rubber occlusor used to initially occlude one prosthesis end represented in 4C.

FIG. 4C is a perspective view of prosthesis in FIG. 4A with elliptical and short intraluminal portions for side-to-side anastomosis, in organs of any gauge.

FIGS. 4D-4M illustrate the execution steps of a side-to-side anastomosis without clamping and sutureless among any type of grafts, in the represented case, in vascular grafts, to avoid the occurrence of bleeding when preparing the anastomosis.

FIG. 5A is a upper view of an oval, rigid and not flexible prosthesis, with more length than width, without intraluminal portion, for anastomosis with grafts of small gauges.

FIG. 5B is an upper view of small gauge graft for anastomosis with oval prosthesis of FIG. 5A.

FIG. 6A is an upper view of an elliptical prosthesis, rigid and not flexible, with more length than width, without intraluminal portion, for anastomosis with grafts of small gauges.

FIG. 6B is an upper view of a small gauge graft for anastomosis with elliptical prosthesis of FIG. 6A.

FIG. 7A is an upper view of an oval, flexible prosthesis, with elastic memory, metallic or not, with more length than width, without intraluminal portion, for anastomosis with grafts of small gauges.

FIG. 7B is an upper view of small gauges grafts for anastomosis with oval prosthesis of FIG. 7A.

FIG. 8A is an upper view of an oval, flexible prosthesis, with elastic memory, metallic or not, with more length than width, without intraluminal portion, for anastomosis with grafts of small gauges.

FIG. 8B is an upper view of small gauges grafts for anastomosis with elliptical prosthesis of FIG. 8A.

FIG. 9A is an upper view of an oval, flexible prosthesis, with elastic memory, metallic or not, with more length than width, without intraluminal portion, for anastomosis with grafts of small gauges.

FIG. 9B is an upper view of small gauges grafts for anastomosis with oval prosthesis of FIG. 9A.

FIG. 10A is an upper view of an oval, flexible prosthesis, with elastic memory, metallic or not, with more length than width, without intraluminal portion, for anastomosis with grafts of small gauges.

FIG. 10B is an upper view of small gauges grafts for anastomosis with elliptical prosthesis of FIG. 10A.

FIG. 11A is a perspective view of a prosthesis variation, with two halves that join by pressure.

FIG. 11B is an upper view of the prosthesis of FIG. 11A before junction.

FIG. 11C is an upper view of the prosthesis of FIG. 11A before junction.

FIG. 12A is a perspective view of a prosthesis variation, with two halves that join by handle.

FIG. 12B is a cutaway perspective view of the prosthesis of FIG. 12A without flange, in order to evidence the handle fixation groove.

FIG. 12C is an upper view of the prosthesis of FIG. 12A before junction.

FIG. 12D is an upper view of the prosthesis of FIG. 12A before junction.

FIG. 13A is a perspective view of a prosthesis variation, with two halves that join by bolt fixation in its bigger opening.

FIG. 13B is an upper view of the prosthesis of FIG. 13A before junction and fixation by bolts.

FIG. 13C is an upper view of the prosthesis of FIG. 13A before junction, in its smaller opening.

FIG. 14A is a perspective view of a prosthesis variation, with intraluminal portion composed by a fixed part and a rocker part, by an adjusting bolt.

FIG. 14B is a perspective view of only the rocker half of the prosthesis intraluminal portion of FIG. 14A.

### DESCRIPTION OF THE PREFERRED EMBODIMENTS OF IMPROVEMENTS

With reference to the detailed description of drawings, in which like reference numbers indicate identical elements throughout the several views, the figures illustrate different forms of the present invention, in the form of a prosthesis for anastomosis with different flange shapes, different shapes of intraluminal portions and different configurations of passing through chamfer openings.

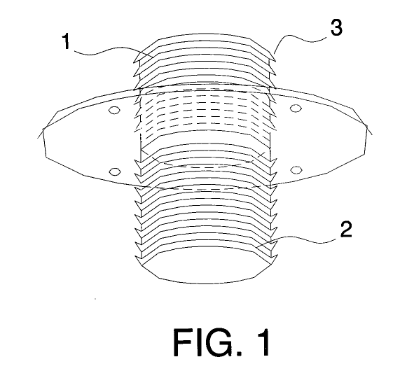

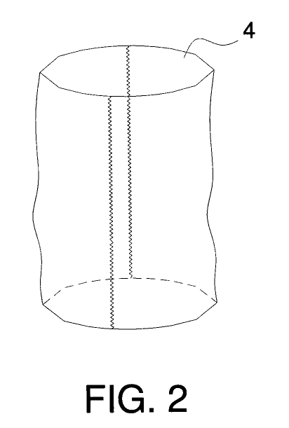

As observed in FIG. 1, a prosthesis with two intraluminal portions is described. Differently from the prosthesis defined in the patent incorporated herein by reference, where only an intraluminal portion was defined, with or without in the other flange end, an edge to avoid the prosthesis light transposition by suture wire, the present prosthesis with a first 1 and a second intraluminal part 2, comprising at least one groove 3 in its circumference, which allow the insertion of grafts of anastomotic trunk 4, as shown in FIG. 2.

Basically, the anastomotic trunk results by junction, by any safe method, for example, seromuscular or total continuous suture of many grafts ends as desired to be joined, in the desired length. The trunk, therefore, will have the same gauge of the sum of the individual grafts gauges, allowing the utilization of only one prosthesis for multiple grafts, providing a single proximal anastomosis, wide, with multiple grafts, at the same type, without clamping and sutureless or, if desired, with expeditious clamping (in organs with normal walls, not friable or calcified), and sutureless.

In total suture, the points transfix all the grafts layers and the wire appears in the internal faces. In seromuscular, the wire includes only the grafts seromuscular layers, but not reaches the inner layer and therefore, it does not appear in the grafts internal surface, and this is the ideal. This would not be performed only if the grafts have the layers is not dissected among them, that is, it would not be fixed to one another in this case, it is mandatory to transfix all the layers in each point, to refixing them, to avoid the fluid flow direction among these layers and not by the grafts light.

The anastomotic trunk can be prepared among grafts of same gauge or different gauges; among grafts with same material and origin or different origin and materials (ex: biological autolog, homolog or heterolog, joints joined among themselves, or synthetic); among grafts of same type or different types (ex: biological type: arterial-arterial, venous-venous or venous-arterial; synthetic type: Gor-Tex-Dacron-PTFE, etc.) etc. This means that the anastomotic trunk can be available previously, sterilized, with biological or synthetic grafts, medusa head type or octopus, where the head is the anastomotic trunk and the members, the individual grafts.

The purpose of the anastomotic trunk is to allow to perform single proximal anastomosis, wide, with multiple grafts, at the same time, without clamping and sutureless with prosthesis, or with clamping and sutureless with prosthesis, or with conventional suture, clamping and without prosthesis. Also, the preparation of anastomotic trunk allows the use of grafts of minimum gauge, which normally is not used due to the technical difficulty and bad results in these small gauge anastomosis.

The prosthesis in FIG. 1 allows the junction of two or more grafts, in any modality (end-to-end, side-to-side or end-to-side, without clamping and sutureless, or with expeditious clamping and sutureless), of different types, gauges and nature, such as autologs, homologs, heterologs or synthetic (Gor-Tex, PTFE, Dacron type, etc.).

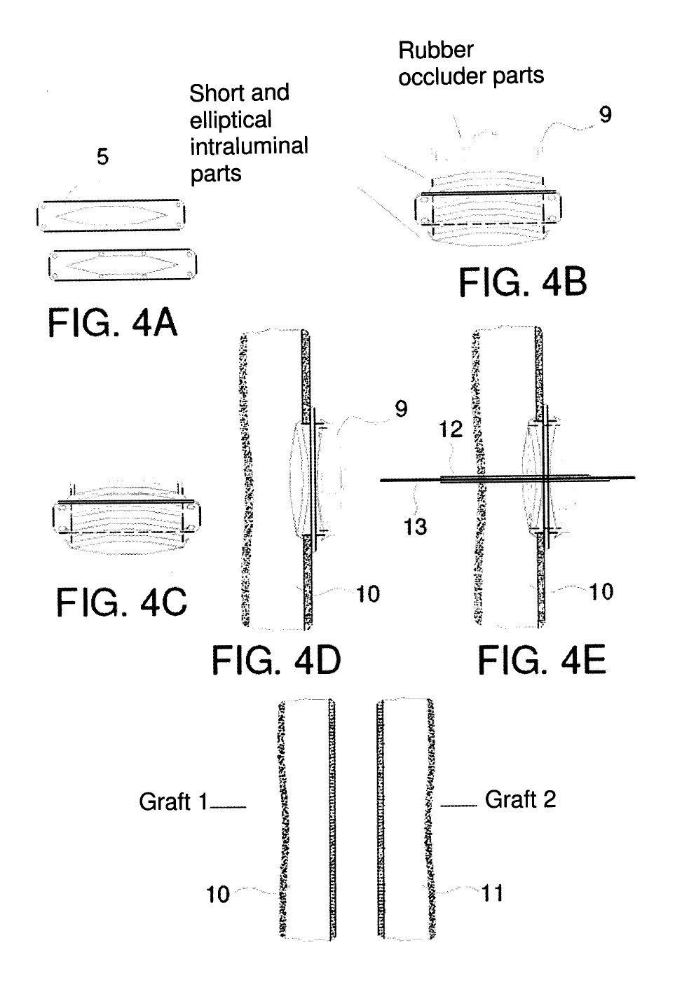

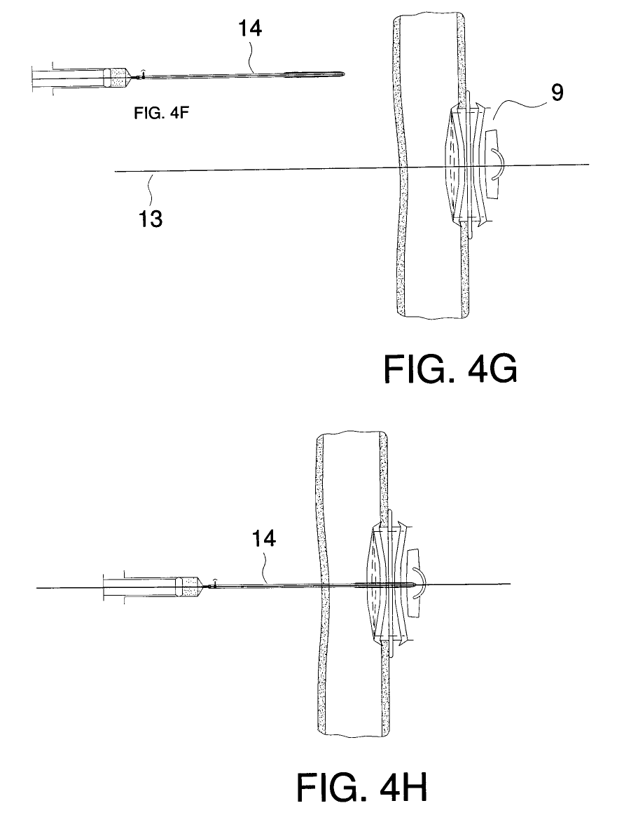

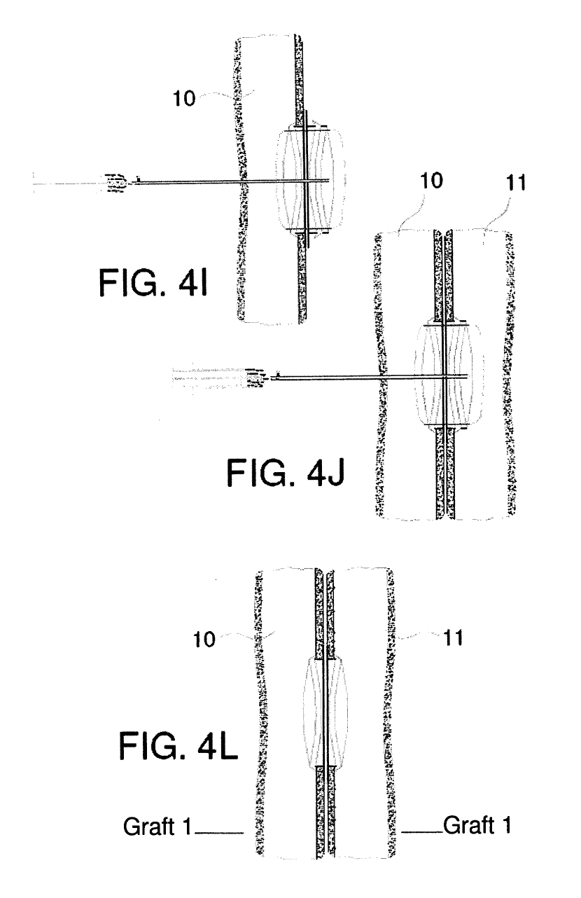

With the prosthesis in FIG. 1, an end-to-end anastomosis without clamping and sutureless could be made following the technique: covering the intraluminal portions with grafts of any nature (if synthetic, it is nonsense to be covered. The prosthesis material being biocompatible and with big gauge is enough. An atraumatic rubber occlusor is used as represented in FIG. 4B, to occlude one end. By the preconized technique of the patent to which this amendment is made, the light of one graft 6 is introduced with a prosthesis end without rubber occlusor. The same procedure is done in the other side and after the incision, which will be temporarily digitally tamponed by an assistant, the prosthesis-graft-occlusor aggregate reaches the incision, the occlusor is removed and the prosthesis graft aggregate is introduced to the other vascular cable light, it is tied, establishing a perfect homeostasis. Then, the two cables are tied with the round point, out of grafts, and proceeding the circumferential ligaments on the prosthesis grafts aggregate inside. So, all the blood flow will be directed and will pass through the prosthesis with double intraluminal portion, covered by grafts such as autolog, homolog, heterolog, synthetic, or not. Other embodiment of the art consists to prior tie vascular cables with a cord or similar wire. After the incision and introduction of the first intraluminal portion of prosthesis, which will be immediately fixed in circumferential form by the cord, the other cable is incised, tamponed with finger, the occlusor is quickly removed and the other intraluminal portion is introduced in the other vascular cable. Also with cord, an extern circumferential fixation is done. The four points preconized in patent PI 9706197-2 passed here. Many other technical modalities, which are out of scope of this description, to be done with the prosthesis in FIG. 1, an end-to-end anastomosis without clamping and sutureless, can also be used.

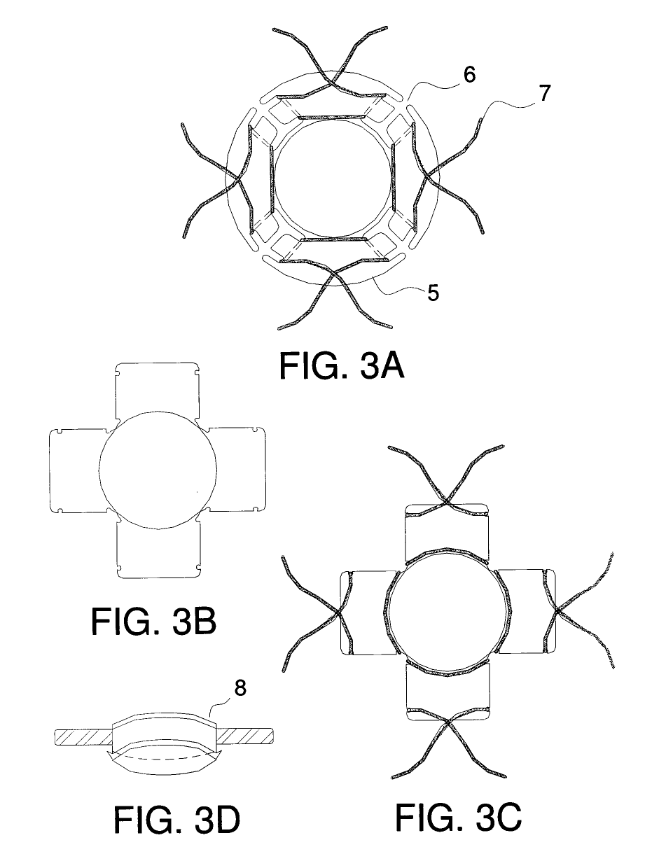

FIG. 3A illustrates the other modality of flange 5, where on the contrary to uniformly arranged passing through holes arranged on peripheral part of the flange, it comprises chamfers 6, where the handle of suture wires 7 is anchored and will be positioned only in chamfers 6, after applying the wires in the organ wall, and immediately before the incision or immediately after the introduction of the prosthesis/graft aggregate under its light. The chamfer 6 shape avoids the handle interposition on the prosthesis light, without the need of edge to anchor them. This flange 5 also avoids the wire handles 7, located in the upper surface cut, cause injury or obstruct the grafts wall that pass through the light.

FIG. 3B also illustrates a flange without holes, petal shaped, with chamfers 6 in the ends. Differs from flange of 3A, because it need the edge 8 (small upward extension, orthogonal to flange, opposed to intraluminal portion), illustrated in FIG. 3D, to anchor the wire handles. With these flanges 5 of FIGS. 3A and 3B, it is easy to make incision and to introduce the prosthesis. After the introduction of the intraluminal portion inside the organ, the wire handles will be placed on flange 5 and the wires 7 will be tied, as illustrated in FIG. 3C.

FIG. 4A illustrates a prosthesis with flanges 5 in rectangular shape in order to inscribe an elliptical intraluminal portion (which biggest diameter is a multiple of the organ diameter) and preferably very short, aiming to perform a side-to-side anastomosis, without affecting the graft lights of small gauge, as shown in FIGS. 4E-4M.

According to technical representation of FIGS. 4A-4M, it was prepared a side-to-side anastomosis without clamping and sutureless with prosthesis of double intraluminal portion, biocompatible, not covered by any type of tissue. The prosthesis with one end closed by rubber occlusor 9, had the other end free, first graft 10 light was introduced by the technique described in the prior patent, in which four points pass, from top to bottom, in holes of prosthesis, applied in point of anastomosis, before the incision. When prosthesis graft aggregate is incised and introduced, the wires are tied, two by two, opposed and in parallel, fixing the prosthesis to organ wall. FIG. 4E represents the punction, with gauge needle 12, of vessel wall, passing through the prosthesis light and transfixing the rubber occlusor 9 that closes the intraluminal portion still not introduced under the light of other vessel. Also in 4E, the guide wire 13 is shown, long that pass through the light of needle 12, which is removed, as represented in FIG. 4G. An aggregate formed by syringe and balloon catheter 14, with central holes, represented in FIG. 4G, it enters backwardly by guide wire 13, and the balloon is directed inside the prosthesis, until reaching the rubber occlusor 9, as represented in FIG. 4H. FIG. 4I represents the inflation of the balloon, occlusing the prosthesis light, and then the guide wire 13 is removed. FIG. 4J represents the continuity of anastomosis, by introducing the other intraluminal portion, occlused by balloon, under the light of the second graft 11, by using the same technique. Finally, the FIG. 4M illustrates the disinflation of the balloon and its removal, followed by a simple points in the wall from where the graft light entered, and the final aspect of the side-to-side anastomosis without clamping and sutureless. This anastomosis can be made in a simplified manner, with expeditious clamping, if the vessel walls are healthy, or in other tubular organ, such as intestines, urethers, esophagus, etc, where clamping or its absence is not so important. Also other technical embodiments can be used.

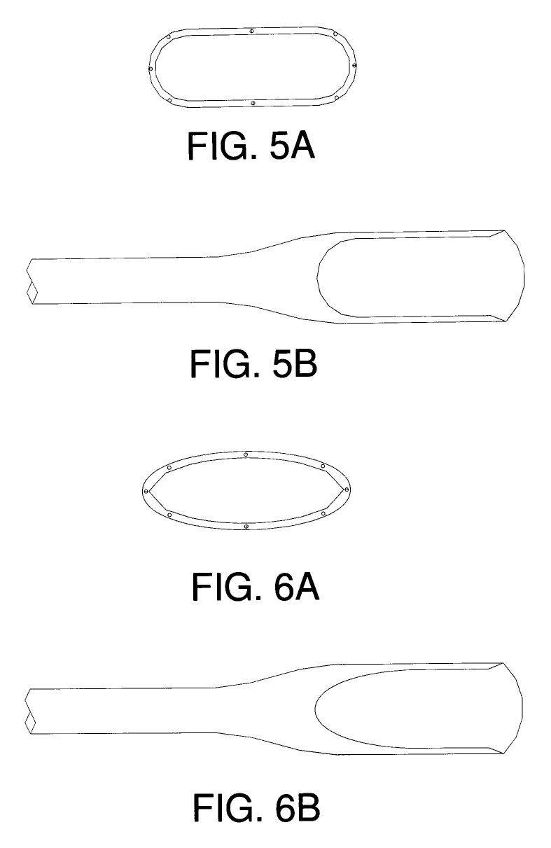

In almost same manner, the FIGS. 5A and 6A illustrate prosthesis with more length than width, with round and elliptical lumens respectively, in rigid material and not flexible, in order to permit one or more grafts (if anastomotic trunk) to be anastomosed with just one prosthesis, performing a wide end-to-side anastomosis, without clamping and sutureless, or with expeditious clamping and sutureless or, if desired, with clamping and with suture. The prosthesis can be first related to distal end of the graft, longitudinally opened, to compatibilize in size with the bigger diameter of the prosthesis, or relating before with another graft side, which will also be longitudinally incised, in equal extension of the prosthesis biggest diameter. These prosthesis have no intraluminal portion or have a minimum intraluminal portion, as a small relief, in order to allow hemostasis, due to the fact that grafts are supported, when tied, by this small intraluminal portion. These figures also show that the holes are interposed by double holes, intended to anchor the wire handles, avoiding the interposition of prosthesis light and its compression, or grafts, from outside to inside, restricting the anastomosis area. Due to the inexistence of intraluminal portion or because these are minimum, it is especially recommended for anastomosis in thin gauge organs such as internal mammary artery with coronary, allowing a wide anastomosis, safe, fast and hemostatic, and can be with or without coronary clamping. In such cases, it must be prepared in a lighter material as possible (ex. titanium, pyrolitic carbon, biodegradable material), with less quantity of material and less weight, in order to avoid compression over these thin gauge vessels. Also, the wires used in anastomosis are more resistant than those of normal use.

These prosthesis with elastic memory, which recover a predetermined original form, or moldable, that keep deformation, especially those without holes but tissue flange, can serve as area keeper for conventional anastomosis, com suture e clamping, from the line of suture the involves it, interposed to two grafts. Thus, the anastomosis will remain open, without collapsing as the conventional, even at pressures equal to zero in the cardiovascular system, or in organs without pressure, such as intestines, urethers, coledoch, uterine tubes, vas deferens, etc.

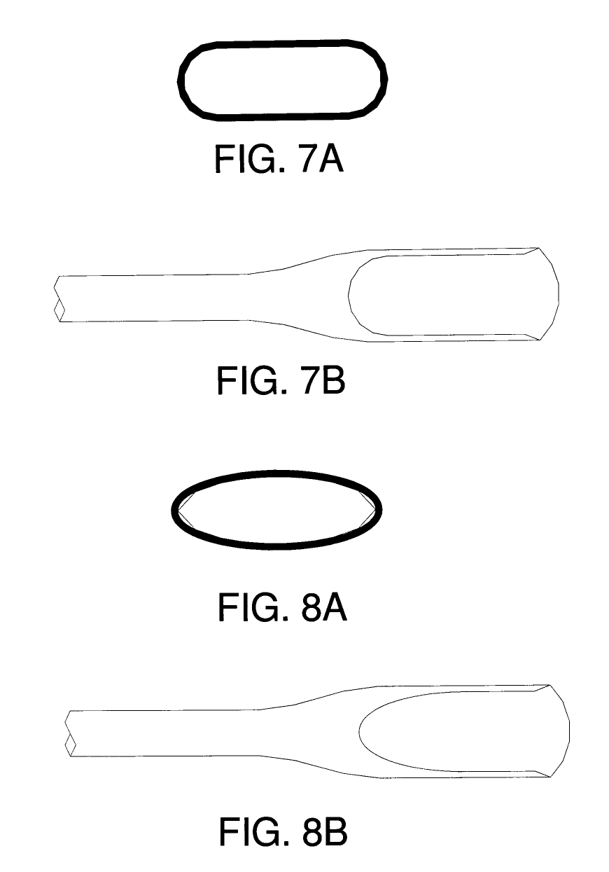

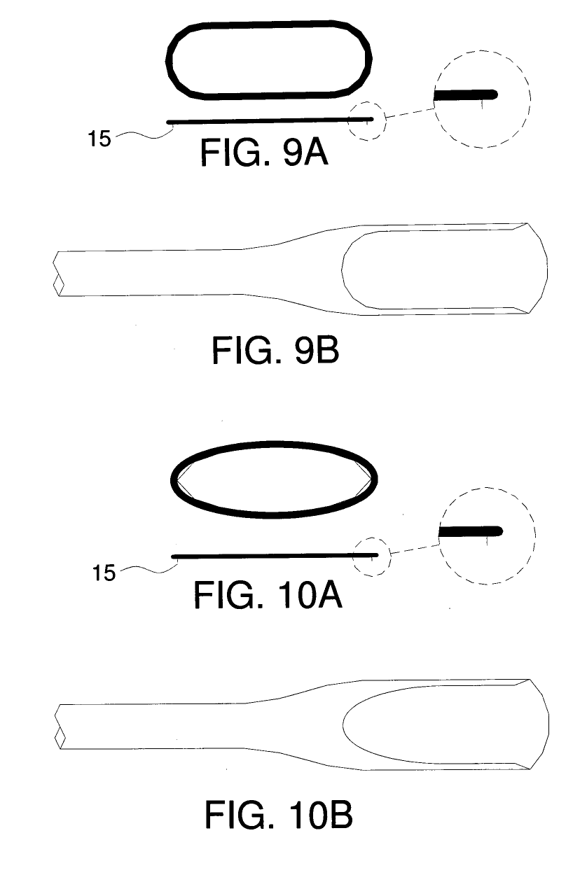

FIGS. 7A and 8A illustrate prosthesis similar to prosthesis of FIGS. 5A and 6A, however, composed by flexible material, with elastic memory or not, and this is moldable as desired at moment of surgery, increasing or diminishing the biggest diameter and respectively reducing or increasing the minor diameter, depending on vessels to be anastomosed, and also without intraluminal portion. If it is flexible and with elastic memory, the anastomosis will have the shape equal to its predetermined form. If the prosthesis is metallic, flexible, with or without elastic memory, it will have holes where the suture wires pass through. In the other hand, if the prosthesis comprises a central wire, metallic or not, with elastic memory or not, but moldable, and this wire is coated by tissue “flange”, there is no need for holes, because the wires will pass and will be anchored in the “flange” tissue. FIGS. 9A and 10A illustrate the same prosthesis with a minimum intraluminal portion 15, in order to assure even more the hemostatic characteristic of the anastomosis, because the grafts are supported and compressed by it.

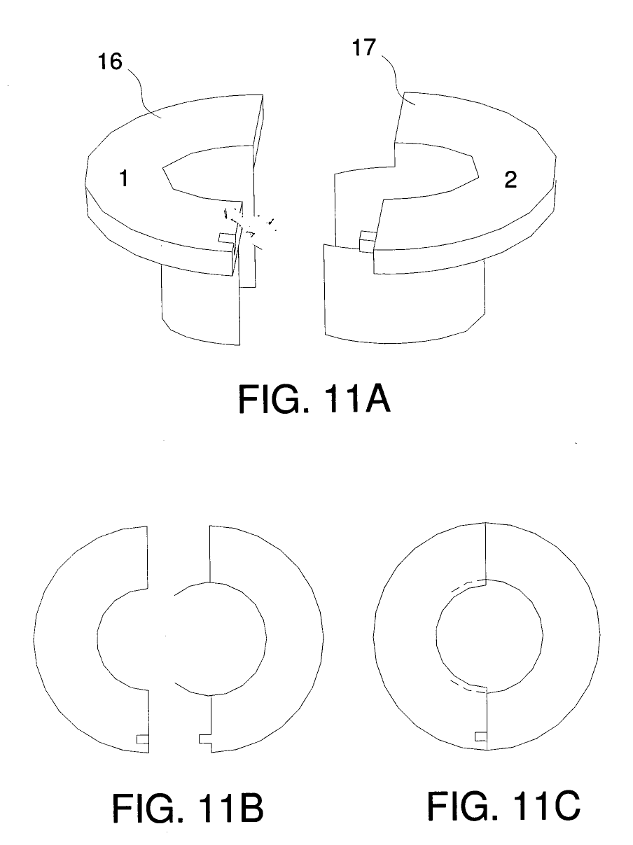

FIG. 11A illustrates a variation of prosthesis with two halves that are joined facilitating the eversion of grafts. The graft is everted on the first half 16. The other half 17 of prosthesis is positioned inside the first half 16 of the already everted graft and after the two halves of prosthesis are joined, fixing it or grafts with external circumferential point. They have holes in the flange and external grooves in intraluminal portion to anchor the everted portion of the grafts, avoiding them to slide after being fixed (neither external grooves nor flange holes were illustrated). In FIG. 11B, the upper view of the halves are still disjoined. The first half 16 (FIG. 11A) has an intraluminal portion with a slightly smaller diameter than the second half 17, in order to facilitate the junction by pressure and to stabilize it, due to the fixation points that does not allow the oscillation of the prosthesis halves upward or downward. Also, an intraluminal portion of the first half 16 has an extension equal to its flange, while the intraluminal portion of the second half 17, has an extension greater than its flange. It is exactly this excess of flange that will be joined by pressure on the other intraluminal portion, with smaller diameter and extension. Besides this, if desired, after the junction (FIG. 11C), the points with suture wire can be applied among flange holes, or round points over the circumferential groove on the flange edge, or over the external grooves of the intraluminal portions, to strengthen these junctions.

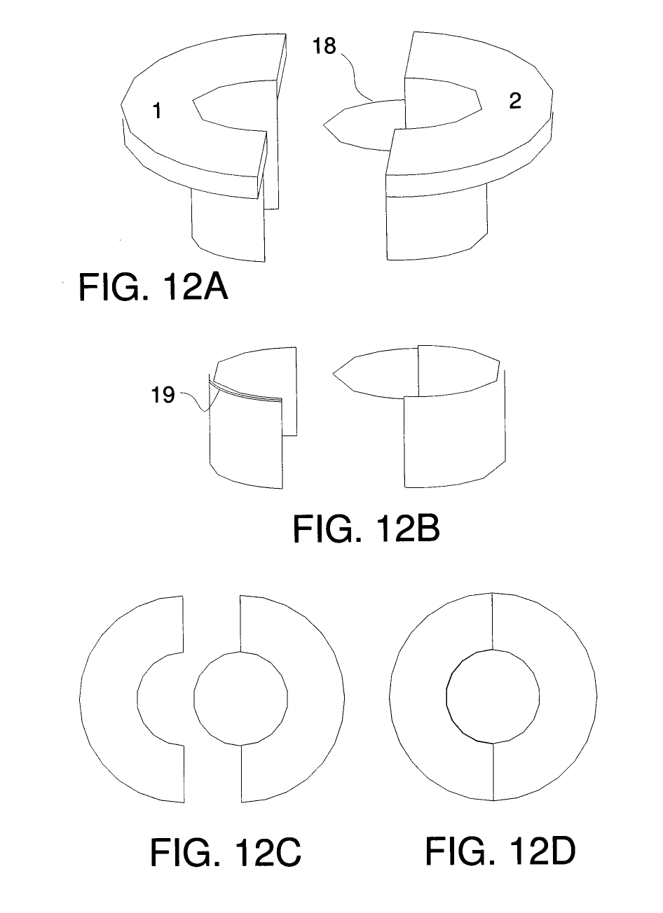

FIG. 12A illustrates a variation of prosthesis with two halves with junction by an elastic handle 18 that is joined in a groove 19 of the first half (FIG. 12B). This also make easier, by the same reason of the prior, the eversion of grafts. But in this case, the graft will necessarily coat the second half that comprises an elastic handle 18, before the first half. Then, the first half will be inserted into the already everted second half of the graft and will be adjusted to the first half when the elastic handle 18 is joined to its groove 19, located immediately lower and adjacent to the flange. Finally, the grafts will be circumferentially fixed to prosthesis with round point. They have holes in the flange and have grooves in intraluminal portion (not illustrated). In FIG. 12C, the still unjoined upper view of halves are shown. The first half 16 has an intraluminal portion of same diameter as the second half 17, and as said, it will remain joined when the elastic handle 18 of the second half 17 is joined into the groove of the first half 16. Also, if desired, after the junction (FIG. 12D), the points with suture wire can be applied by the flange holes, by circumferential groove in flange edge or in external grooves of intraluminal portion, to strengthen these joints.

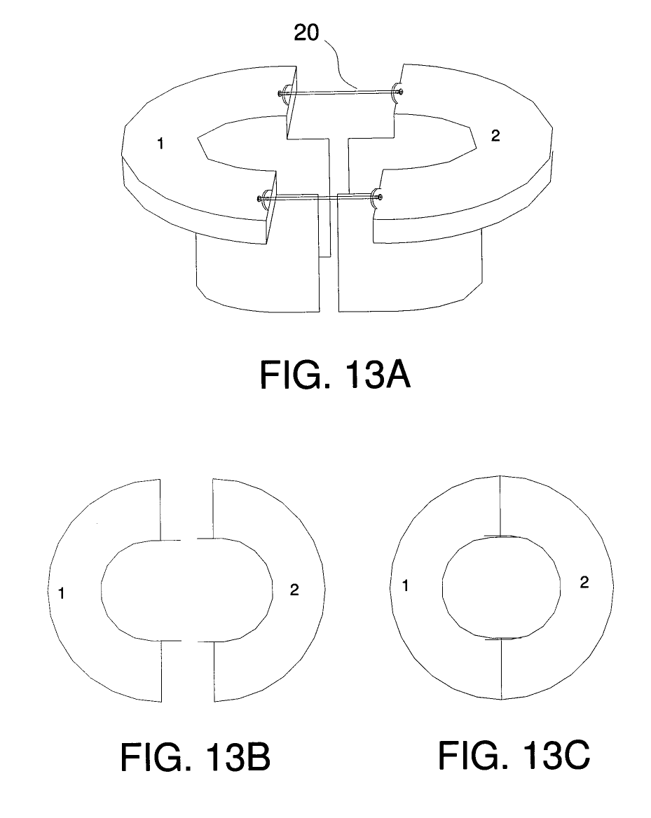

As a different embodiment, FIG. 13A illustrates a prosthesis with two halves, with a distance among them adjusted by a fixation device 20, for example, a bolt, presenting in its greatest diameter as possible, where the intraluminal portions are separated from one another. These prosthesis also allows the eversion of grafts and permits the use of grafts of varied diameters. It can be said “universal prosthesis” (establishing the minimum maximum limit of grafts diameters that could be used with it, or by joining them in an anastomotic trunk, until reaching the compatible diameter with its biggest diameter). The eversion of any graft or anastomotic trunk which diameter is greater than the smaller diameter of the prosthesis, is very easy. When adjusted to the smaller diameter, the bigger graft, or anastomotic trunk pass through the light and without complexity, it will be everted, covering the intraluminal portions, being fixed to them, with an external circumferential point. They have holes in the flange and grooves in the intraluminal portion (not illustrated). All these prosthesis have blind, without cut, round and atraumatic flange edges and intraluminal portion. They must be prepared in biocompatible material (stainless steel, titanium, nitinol, pyrolitic carbon, gold, silicon, PTFE, DACRON, GOR-TEX etc, or any biodegradable material), with less quantity of material as possible, maintaining their characteristics. In FIG. 13B is shown the upper view of the still unjoined halves, where the intraluminal portions with slightly different diameters, to allow them to roll over one another, increasing or diminishing the prosthesis diameter, changing its shape, making it very versatile and can perfectly accommodate with single prosthesis, grafts or anastomotic trunks of variable gauges. FIG. 13C represents this prosthesis joined in its minor diameter.

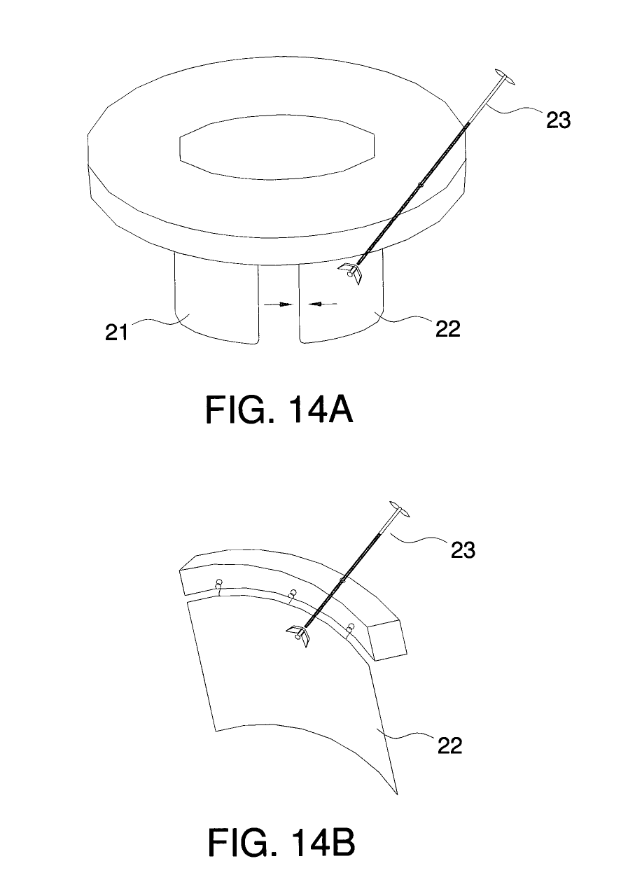

FIG. 14A shows another variation of an adjustable prosthesis, with the intraluminal portion divided in two elements: fixed 21 and rocker 22 by an adjusting device 23, for example, a bolt, that transfix the external flange, facilitating the eversion of the graft, due to the diameter variation, also allowing the use of grafts or anastomotic trunks of variable gauges. The flange holes or the external grooves of the intraluminal portion were not represented. In FIG. 14B, the rocker half 22 of the intraluminal portion is shown, with the removable adjusting bolt 23 placed on the upper portion, which when activated, increases or diminishes the prosthesis diameter.

1. Prosthesis to anastomosis comprising: a tubular member having a first intraluminal portion (1), a lumen and a flange (5) that extends from a side wall of the tubular member, wherein the flange (5) has a plurality of through chamfers (6) distributed over the surface; and the tubular member also comprising a second intraluminal portion (2) to joint at least one graft.

2. Prosthesis, according to claim 1, wherein the first and second intraluminal portions (1,2) comprise at least one groove (3) on an external surface of the side wall, in a transversal plan direction of the lumen of the tubular member.

3. Prosthesis, according to claim 1, wherein the first and second intraluminal portions (1, 2) join at least two grafts of equal or different sizes and types.

4. Prosthesis, according to claim 1, wherein the flange and intraluminal portions have a plurality of shapes, preferably a circular, oval or elliptical shape.

5. Prosthesis, according to claim 1, wherein the prosthesis is formed by two halves (16, 17).

6. Prosthesis, according to claim 5, wherein the halves (16, 17) are joined by pressure and joining points to stabilize it.

7. Prosthesis, according to claim 5, wherein the halves (16, 17) are joined by at least one fixation device (20).

8. Prosthesis, according to claim 5, wherein the halves are moved towards or away from one another by an adjusting device (23).

9. Prosthesis, according to claim 8, wherein an intraluminal half is fixed (21) and the other is rocker (22).

10. Prosthesis according to claim 8, wherein the adjusting device (23) is a bolt.

11. Prosthesis for anastomosis wherein the flange is rigid and is longer than it is wide with through openings in a peripheral portion.

12. Prosthesis, according to claim 11, wherein the flange can be circular, oval or elliptical.

13. Prosthesis for anastomosis, comprising a flexible device with elastic memory, that is longer than it is wide, without openings passing through its peripheral portion.

14. Prosthesis for anastomosis, comprising a flexible device, moldable, without elastic memory, that maintains a deformation to which it has been subjected.

15. Prosthesis, according to claim 12, further comprising a minimum intraluminal portion (15).

16. Prosthesis, according to, claim 13 wherein the flange can have a circular, oval or elliptical shape.
